(19) United States
(12) Patent Application Publication
(10) Pub. No.: US 2007/0042352 A1
(43) Pub. Date: Feb. 22, 2007

(54) Retroviral vector and cell-based assay for measuring the mutations rate of retroviruses employing same

(76) Inventor: Dawn Patricia Wooley, Beavercreek, OH (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 10/569,159

(22) PCT Filed: Aug. 18, 2004
(86) PCT No.: PCT/US04/26787

§ 371 Date: Oct. 13, 2006

Related U.S. Application Data
(60) Provisional application No. 60/496,333, filed on Aug. 18, 2003.

(51) Int. Cl. C12Q 1/70; C12N 15/867

(52) U.S. Cl. 435/5; 435/456; 435/325

(57) ABSTRACT

Lentiviral-based retrovirus vectors and an in vivo mutation rate assay employing them. More particularly, an assay for directly determining the in vivo mutation rate of HIV-1.

The present invention is directed toward a cell-based assay that allows direct measurement of the mutation rate of retroviruses during replication. The assay employs a novel lentivirus-based retroviral vector which is replication defective but transduction and infection competent. More particularly, the assay may be used to calculate the mutation frequency and mutation rate of HIV-1.

Over twenty years into the ever-worsening AIDS pandemic, genetic variation remains the greatest obstacle for treating and preventing HIV-1 infection. To date, there are no assays that directly measure the mutation rate of HIV-1 during replication in cell culture. There is an established need in the art for a phenotypic cell-based test for directly measuring the mutation rate of HIV-1.

A high degree of genetic variation is associated with retroviruses in general and HIV-1 in particular, during the course of infection. This variation enables the virus to escape the host immune response, use multiple cell surface proteins for viral entry, mount resistance to antiretroviral drugs, and prevent effective vaccination. The polymerase enzymes responsible for replicating the HIV-1 genome are host cell DNA-dependent DNA polymerase, host cell RNA polymerase II, and the virally-encoded reverse transcriptase enzyme (RT). The host cell DNA-dependent DNA polymerase replicates the integrated provirus during cellular proliferation; it is not a mutation-prone polymerase and has a negligible contribution to the high mutation rate during HIV-1 replication. The host cell RNA polymerase II is responsible for transcription of the integrated provirus. To date, an accurate measurement of the RNA polymerase II mutation rate has not been reported. However, evidence suggests that RNA polymerase II has proofreading capability, diminishing its reputed role in HIV-1 mutation. Reverse transcriptase (RT) copies the single-stranded RNA genome into a double-stranded DNA molecule. The RT enzyme is notoriously error-prone; its mutation rate is several orders of magnitude higher than DNA polymerases, due mainly to the lack of an associated 3′→5′ exonuclease activity. Consequently, RT is considered to be the principal contributor to HIV-1 genetic variation.

Early in the AIDS epidemic, measurements of the HIV-1 mutation rate were performed in cell-free studies using artificial templates and purified enzyme and substrates (hereafter referred to as in vitro studies). These in vitro studies predicted the mutation rate for HIV-1 to be on the order of 10⁻⁴ mutations per base per replication cycle. Other in vitro studies followed these initial reports using various assays to characterize the types of mutations such as base substitutions, insertions, and deletions that are associated with the high RT mutation rate. In vitro systems, however, may not accurately duplicate the physiologic conditions of a replicating virus. For example, retroviral mutations rates are higher in vitro as compared to rates measured during replication of the virus in cells (hereafter referred to as in vivo). Other researchers have shown that in vitro systems can generate reverse transcription products that are not naturally found in vivo, such as extended minus-strand strong stop DNA and non-templated base additions.

In 1995, Mansky and Temin, (J. Virol. 689:5087-94 (1995)), reported the development of an in vivo assay for measuring the mutation rate of HIV-1. The Mansky assay, however, measures the mutation rate indirectly and has several major drawbacks. The assay is indirect because it does not detect mutant proviruses in the target cell. Instead, the mutational target sequence, the lacZa peptide gene, is excised from the provirus and introduced into a bacterial system, where the mutational screening actually takes place. The screening is based on visual blue/white color selection of bacterial colonies on agar plates. This color selection is not an all-or-none effect. That is, there are many intermediate shades of blue, creating a subjective mutation detection process. The lacZa peptide gene is isolated from a sea of pooled target cell genomic DNA by binding to a Lac repressor protein and recovery on nitrocellulose. This method is very labor intensive. Furthermore, the Lac repressor protein is not readily available and typically must be custom-made. The reference assay is also indirect because colonies on the target cell dishes are pooled after the single cycle of viral replication. This pooling step results in a sampling type of measurement as opposed to a direct measurement. There is no way to know whether mutants are independent, potentially skewing the results on the types of mutations observed in this system. Because the cells are pooled, there is also no way to know the total number of viruses screened or number of mutant viruses detected. The lacZa peptide gene is small (280 bp) and the viral titers in the reference assay are low. These two features decrease the frequency by which mutants are detected and increase the number of infections that must be performed, thus increasing cost. In fact, in order to achieve sufficient viral titers, the reference assay relies on co-cultivation of mitomycin-treated virus-producing cells with target cells. Therefore, the vast majority of viral infections are forced cell-to-cell as opposed to infections by cell-free virus particles. The last major drawback of this assay is that it has not been made readily available to other researchers for independent verification and scientific study.

Hence, there is a need in the art for a more economic, less-labor intensive, improved, readily available and independent cell-based assay to directly and more accurately measure the mutation rate of retroviruses, and, in particular, HIV-1.

Accordingly, embodiments of the present invention are designed to provide a mutation rate assay that overcomes one or more of the deficiencies discussed above.

In a broad vector embodiment, a lentivirus-based retroviral vector is provided wherein the vector comprises: at least a portion of a lentivirus genome; a disrupted gag and a disrupted pol gene such that the vector is rendered replication-defective; a disrupted env gene; and a mutational cassette, wherein the mutational cassette comprises sequences encoding: a mutation target promoter sequence; a genomic source comprising a mutation target gene wherein there is a number of base pairs in the mutation target gene; an internal ribosome entry site for expression of a selectable marker; and a selectable marker gene.

In more specific embodiments, the lentivirus genome is selected from a group of retroviruses including human immunodeficiency virus type 1. In other specific embodiments the selectable marker comprises a hygromycin B resistance gene, the mutation target promoter comprises a human cytomegalovirus promoter, the mutation target may comprise a thymidine kinase gene, and the genomic source comprising a mutation target gene may comprise a human herpes virus type 1 gene. Another aspect of the invention is directed to a cell comprising the inventive lentivirus.

Another embodiment of the invention is directed to an assay for determining mutation frequency and mutation rate of a retrovirus. The assay comprises: a) constructing a pertinent embodiment of the inventive vector; b) stably transfecting cells from a cell culture with the vector from (a) wherein the cell culture is negative for the mutation target gene; c) placing the cells under selection with a medium selectable for the selectable marker to produce a quantity of cell clones which contain an integrated vector; d) transiently transfecting the quantity of cell clones with a set of helper plasmids to produce a vector virus, wherein the set of helper plasmids contain a complement of structural genes which permit replication; e) infecting naïve cells from a cell culture with the vector virus; f) placing the cells from (e) under selection with the medium selectable for the selectable marker; g) cloning the cells from (f) to produce a quantity of initiator cell clones, wherein each of the quantity of cell clones is designated as an Initiator Clone (IC); h) confirming that the mutation target gene is functional in each IC and sequencing the mutation target gene for later comparison to mutation target genes which have undergone a cycle of replication; i) transiently transfecting the ICs with a set of helper plasmids to produce a vector virus; j) infecting naïve cells from a target cell culture with the vector virus from (i) to produce a quantity of infected target cells; k) placing a first portion of the quantity of infected target cells under selection with the medium selectable for the selectable marker, placing a substantially similar second portion of the quantity of infected target cells under selection with the medium selectable for the selectable marker plus a selective medium for the mutation target gene; and l) determining a viral titer for the first portion and a viral titer for the second portion by counting drug-resistant quantities, wherein the mutation frequency is calculated by dividing the viral titer of the second portion by the viral titer of the first portion, and the mutation rate is calculated by dividing the mutation frequency by the number of base pairs in the mutation target gene.

In specific embodiments the retrovirus is selected from the group consisting of, inter alia, human immunodeficiency virus type 1. In further specific embodiments the selectable marker gene may comprise a hygromycin B resistance gene, the medium selectable for the selectable marker may comprise hygromycin B, the mutation target gene may comprise thymine kinase, and the medium selectable for the mutation target gene may comprise bromodeoxyyuridine.

Embodiments of the novel assay have one or more advantages and/or improvements over other known in vivo mutation rate assays. It is easier, more economical, and faster to perform. It provides a direct measurement of the mutation rate during a single cycle of viral replication, a known number of mutants detected, and a known number of viruses screened. The inventive assay incorporates a larger target sequence that yields higher mutation frequencies and uses a natural viral infection process (as opposed to forced cell-to-cell infection). The present assay also has the potential for mechanization.

The present retroviral vector and assay are additionally advantageous in the field of personalized medicine because they provide the potential for individualized monitoring of drug regimens in HIV-infected patients and for monitoring the development of drug resistance in infected individuals as well as across infected populations. The resultant target cells containing mutant proviruses that survive the drug selection process and may be propagated for further analysis. The vector and assay may be employed to study genetic variation of retroviruses including HIV-1.

Embodiments of the assay described herein are useful for studying HIV-1 evolution with respect to drug and vaccine development. In addition, embodiments of the assay may provide a novel phenotypic test that can measure the rate at which HIV-1 is mutating. Additional embodiments, objects and advantages of the invention will become more fully apparent in view of the following detailed description an in conjunction with the accompanying drawings.

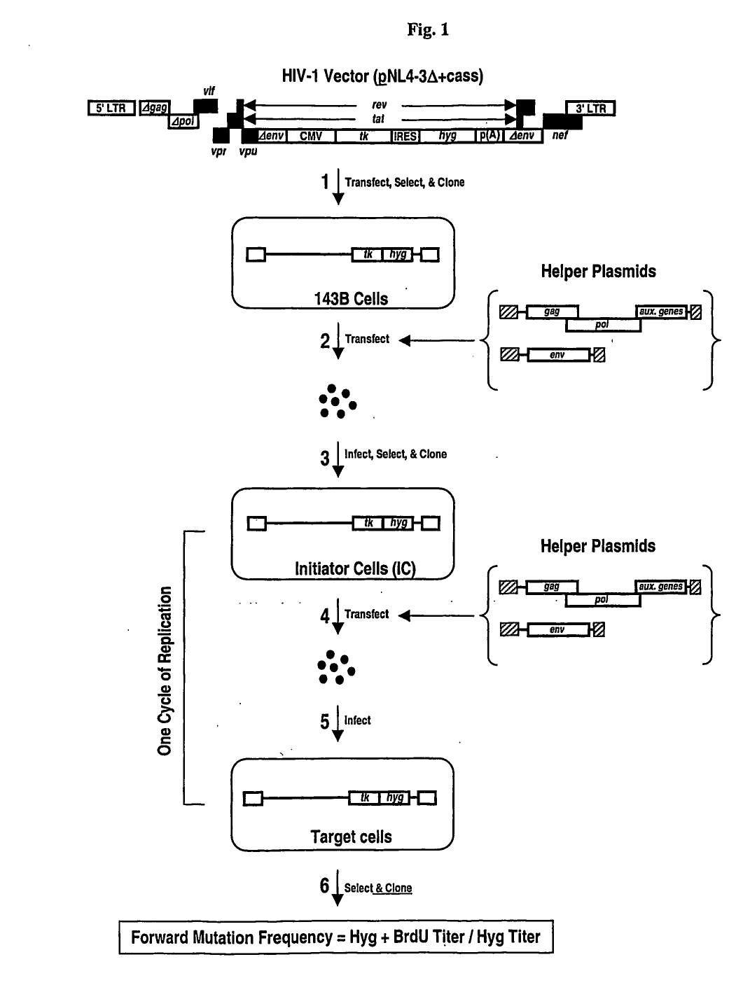

FIG. 1: depicts an HIV-1 vector and flow chart for an embodiment of the inventive mutation assay. The HIV-1 vector, pNL4-3Δ+cass, is shown at the top. Step 1: The vector is stably transfected into naïve 143B cells. The cells are placed under selection with hygromycin B (Hyg) and cloned. Step 2: The 143B cell clones containing an integrated HIV-1 vector are transiently transfected with HIV-1 helper plasmids to produce vector virus. Step 3: Vector virus is used to infect fresh 143B cells. The cells are placed under selection with Hyg and cloned. Step 4: The 143B cell clones containing an integrated HIV-1 vector are transiently transfected with HIV-1 helper plasmids to produce vector virus (same as step 2 except cells are infected, not transfected). Step 5: Vector virus is used to infect fresh 143B cells. Step 6: Parallel infections are placed under selection with Hyg alone and Hyg plus BudR. Viral titers are calculated by counting the number of drug resistant colonies. The forward mutation frequency is calculated as shown. Cells are cloned for further analysis.

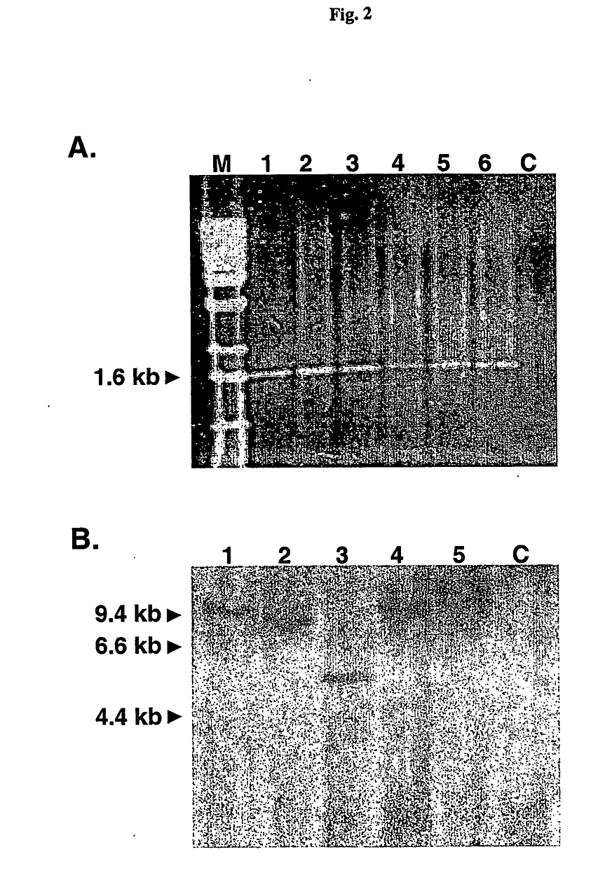

FIG. 2: depicts an analysis of the Initiator Clones (ICs). (A) Agarose gel showing PCR results performed on genomic DNA of ICs 1-6 and genomic DNA of 143B cells. The 1.6 kb band is specific for the HIV-1 vector sequence spanning CMV through TK. Lane M is a 1 kb marker. The predicted 1.6 kb PCR product was detected in all ICs but not in 143B genomic DNA, as expected. (B) Southern blot analysis of ICs 1-5. Lane C represents the 143B genomic DNA negative control. A high molecular weight marker was included on the gel with sizes listed on the left side. Only one band was detected for each IC, indicating one provirus per cell. In addition, the bands for each IC were different sizes, indicating that the ICs were independent cell clones.

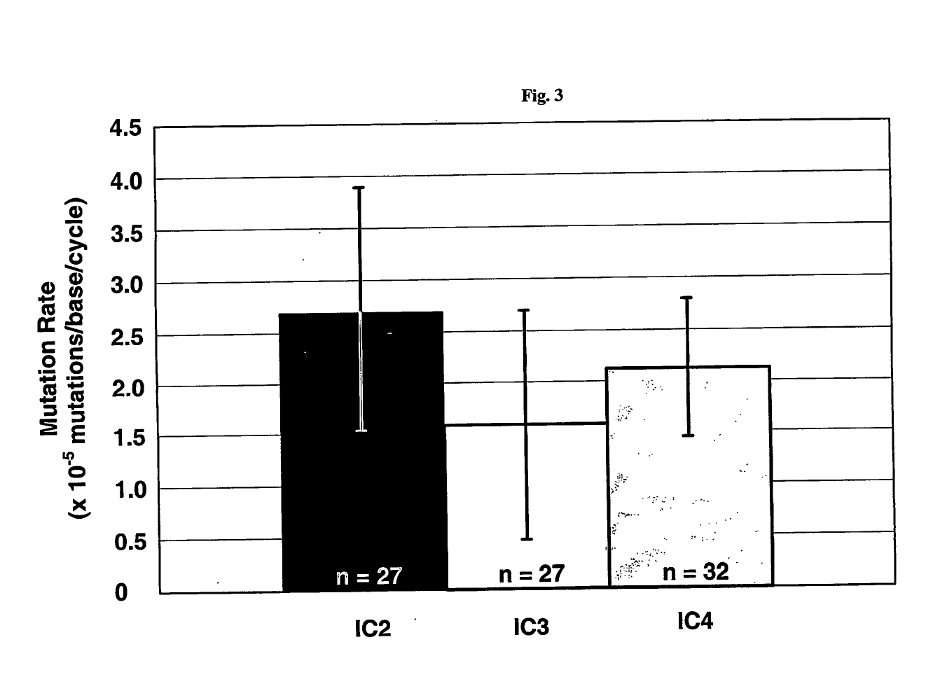

FIG. 3: illustrates the mutation rate of HIV-1. The mutation rate for HIV-1 was measured from three independent Initiator Cell clones (ICs). The number of infections is listed inside the column for each IC. The error bars represent 1 standard deviation from the mean.

The following definitions will be useful to understand the several embodiments of invention disclosed herein.

“cis”—refers to the presence of genes on the same chromosome, viral genome, or molecule; cis-acting—used in reference to the controlling effect of a regulatory gene or element on a gene present on the same chromosome, viral genome, or molecule. Promoters, which affect the synthesis of downstream mRNA are cis-acting control elements.

Retrovirus—RNA viruses that utilize reverse transcriptase (RT) during their replication cycle. Retroviral genomic RNA is converted into double-stranded DNA by RT. The double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles. At each end of the provirus are structures called “long terminal repeats” or “LTRs”. The LTR contains numerous regulatory signals including a promoter.

Lentivirus—a genus in the Retroviridae family of retroviruses that give rise to slowly developing disease. Diseases caused by these viruses are characterized by a long incubation period and protracted course. An important factor in the disease caused by these viruses is the high mutability of the viral genome, which, inter alia, results in the production of mutants capable of evading the host immune response. Non-limiting examples employable in the present invention include HIV-1, HIV-2, visna-maedi virus, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immunodeficiency virus (BIV), which causes lymphadenopathy, lymphocytosis, and possibly central nervous system infection in cattle; and simian immunodeficiency virus (SIV), which cause immune deficiency and encephalopathy in sub-human primates.

HIV (human immunodeficiency virus; including HIV-1, and HIV-2), the etiologic agent of the human acquired immune deficiency syndrome (AIDS).

Gene—a DNA sequence that comprises control and coding sequences necessary for the production of a particular protein, polypeptide or precursor—any portion of the coding sequence so long as the desired enzymatic activity is retained. A unit of heredity.

Mutant—a gene or gene product which displays modifications in sequence and or functional properties when compared to the wild-type gene or gene product.

Replication defective—refers to a virus that is not capable of a complete, effective replication cycle such that infective virions are not produced.

Provirus—used in reference to a virus that is integrated into a host cell chromosome (or genome) and is transmitted from one cell generation to the next without causing lysis or destruction of the host cell.

Selectable marker—a gene which encodes a protein with a function that allows for the identification of cells that have been transfected or transformed with the marker gene (for example, an enzyme that confers resistance to an antibiotic or drug upon the cell in which the selectable marker is expressed). A positive selective marker is a gene whose expression permits the cell to live in the presence of a selectable agent. The selectable agent is a compound that distinguishes cells that do not express the selectable marker, typically by killing them. Bacterial hygromycin B phosphotransferase (hyg) that confers resistance to the antibiotic hygromycin B and the bacterial xanthine-guanine phosphoribosyl transferase gene (also referred to a the gpt gene) that confers the ability to grow in the presence of mycophenolic acid are examples. Other selectable markers negative in that their use kills cells that do express the protein encoded by the selectable marker and their use is typically in conjunction with a cell line that lacks the relevant activity. Examples of negative selectable markers include the thymidine kinase (tk) gene that is used in conjunction with TK-negative cells. It is understood that TK-negative means that the cell does not express a functional TK protein and does not necessarily mean an absolute absence. A person skilled in the art will appreciate that there are many manipulations and disablements that may be undertaken to eliminate the expression of a functional form of the typically expressed protein.

Vector—nucleic acid molecules that transfer nucleic acid segments from one cell to another. It is intended that any form of a vector may be encompassed by this definition. For example, vectors include but are not limited to: viral vectors; plasmids; transposons; and so on.

Cassette—a fragment or segment of DNA containing a particular grouping of genetic elements and/or genes. A cassette can be removed and inserted into a vector or plasmid as a single functional unit.

Transfection—the uptake, expression and/or incorporation of foreign DNA into eukaryotic cells. May be accomplished by a variety of means known or yet to be developed in the art.

Transduction—refers to the delivery of genetic material using a viral or retroviral vector by means of infection rather than by transfection. In specific embodiments, retroviral vectors are used to transduce eukaryotic cells. E.g. a gene carried by a retroviral vector can be transduced into a cell through infection and provirus integration. A transduced gene is one that has been introduced into the cell via lentiviral or vector virus infection and provirus integration.

Stably transduced—refers to the introduction and integration of foreign DNA into the genome of the transducted cell. “Stable transductant” refers to a cell which has stably integrated foreign DNA into the genomic DNA.

Transiently transduced—introduction of foreign DNA into a cell where the foreign DNA fails to integrate into the genome of the transducted cell. The foreign DNA may persist in the nucleus of the transducted cell for several days. During this time the foreign DNA is subject to the regulatory controls that govern the expression of endogenous genes in the chromosomes.

Non-dividing cell—target and/or host cells that do not divide, e.g. neuronal cells. Embodiments of the present assay are not intended to be limited to non-dividing cells.

Embodiments of the present inventive lentiviral-based retrovirus vector and cell-based assay for measuring the mutation rate of retroviruses are described in detail below.

The inventive assay as exemplified herein will yield conservative measurements for mutation rates. First, the entire tk sequence (996 bp) is used in the calculation. Second, some mutant proviruses will have more than one mutation. Although every base in the tk gene is subject to a potential deletion or insertion, not every base is subject to a non-synonymous base change. For tk, the probability of whether a base substitution will result in an amino acid replacement is 74%. That is, if random mutations occurred throughout the gene, 737 out of the 996 base changes would result in an amino acid change, but not all amino acid substitutions would result in loss of function for TK. Amino acid substitutions that do not knock out TK function would be susceptible to selection by BudR and would not be detected in the assay. The inventors are not aware of any reports on saturation mutagenesis of tk. Therefore, a comprehensive catalog of inactivating tk mutations is not available. If the mutation rate in this study had been calculated using 737 instead of 996 bases for tk, then the mutation rate would be 3.0×10⁻⁵/base/cycle. The decision to include all 996 bases in the calculation considers all types of mutations: deletions, insertions, and substitutions. Mutant proviruses were counted as one in the mutation rate calculation, even though some mutants contained multiple genetic alterations. Table 2, below, shows that there were 43 mutations among 27 mutants screened. If the mutation rate was adjusted to account for multiple mutations, then the rate would be 3.5×10⁻⁵ mutations/base/cycle. Thus, as stated, the reported mutation rate is a conservative one and could be as much as 1.6-fold higher.

One embodiment of the inventive vector is directed to a lentivirus-based retroviral vector wherein the vector comprises: at least a portion of a lentivirus genome; a disrupted gag and a disrupted pol gene such that the vector is rendered replication-defective; a disrupted env gene; and a mutational cassette, wherein the mutational cassette comprises sequences encoding: a mutation target promoter sequence; a genomic source comprising a mutation target gene wherein there is a number of base pairs in the mutation target gene; an internal ribosome entry site for expression of a selectable marker gene; and a selectable marker gene.

Retroviruses are a class of viruses that have a single stranded, positive sense RNA genome and replicate through a double-stranded DNA intermediate directed by the process of reverse transcription (RT). The DNA intermediate is referred to as the provirus. The provirus integrates into the host cell genome, thereby becoming acquired genetic material. HIV-1 is a lentivirus, which is a genus of retroviruses. Lentiviruses are unique retroviruses that possess the ability to replicate in quiescent cells as well as replicating cells. Lentiviruses are associated with immune deficiency and neurodegeneration of the host. Examples of other lentiviruses include simian immunodeficiency virus (SIV), bovine immunodeficiency virus (BIV), and feline immunodeficiency virus (FIV).

A gene is “disrupted” if it is rendered incapable of expressing its functional polypeptide. It may be disrupted by completely deleting it or by removing its initiation codon, inserting stop codons or base pairs to force a frameshift. One skilled in the art will appreciate that there are many effective means to disrupt a gene. In a specific embodiment the vector is replication deficient but transduction- and infection-competent.

In another specific embodiment the lentivirus genome is selected from the group consisting of human immunodeficiency virus type 1 (HIV-1), human immunodeficiency virus type 2, feline immunodeficiency virus, simian immunodeficiency virus, visna-maedi virus, caprine arthritis-encephalitis virus, equine infectious anemia virus, and bovine immunodeficiency virus. In a very specific embodiment the lentivirus genome comprises HIV-1. In certain embodiments the genome may comprise a hybrid of the aforementioned lentivirus genomes.

In one aspect of the retrovirus vector the selectable marker comprises a positive selectable marker. In a further aspect the selectable marker comprises an antibiotic or drug resistance gene. In a very specific aspect, the internal ribosomal entry site promotes translation of the selectable marker and the selectable marker comprises a hygromycin B resistance gene. One skilled in the art will appreciate that the choice of promoter depends upon which drug resistance gene is employed as the selectable marker and that many means are commonly known in the art that suggest or determine an appropriate selection.

In specific embodiments the number of base pairs in the mutation target gene is greater than 300 base pairs. A greater number of base pairs increases the size of the mutational target and, therefore, increases the statistical reliability and validity of the results. In more specific embodiments the number of base pairs in the mutation target gene is greater than 500 base pairs, and in a very specific embodiment the number of base pairs in the mutation target gene is greater than 700 base pairs. In a particular embodiment the mutation target comprises a thymidine kinase gene that comprises about 1000 base pairs. In one embodiment the genomic source comprising a mutation target gene comprises a human herpes virus type 1 gene.

Promoters are used to enhance or increase the expression of genes in the retrovirus vector. A promoter is a regulatory element that facilitates the initiation of transcription of an operably linked coding region. The selection of a particular promoter depends on what cell type is to be used to express a protein, polypeptide or enzyme of interest. In one particular embodiment the mutation target promoter comprises a human cytomegalovirus (CMV) promoter and the mutation target comprises a thymidine kinase (tk) gene.

Another embodiment of the invention is directed to a cell comprising the present lentivirus-based retroviral vector. The cell may comprise any cell which the vector is capable of infecting. Generally speaking, it will be a eukaryotic cell, preferably a vertebrate cell, more preferably a cell of a mammal. The cell may be a dividing or non-dividing cell. Non-limiting examples of non-dividing cells include neuronal cells and astrocytes. Non-limiting examples of dividing cells include hematopoietic stem cells, muscle cells, white blood cells, spleen cells, liver cells, epithelial cells and eye cells. In one embodiment the cell of interest is negative for the mutation target gene. It is appreciated by those skilled in the art that “negative” in this context does not necessarily mean it is not present, but that it is disabled or disrupted to a degree that prevents the expression of a functional expression product. The inventors contemplate that expression product functioning may even be suppressed by an agent such as a drug or other compound. In a particular embodiment where the mutation target gene is thymidine kinase, the cells comprise 143B cells. In one particular embodiment the cell is a dividing or non-dividing eukaryotic cell.

Another embodiment of the present invention provides an assay for determining a mutation frequency and a mutation rate of a retrovirus. The assay comprises: a) constructing the vector described above; b) stably transfecting cells from a cell culture with the vector from (a) wherein the cell culture is negative for the mutation target gene; c) placing the cells under selection with a medium selectable for the selectable marker to produce a quantity of cell clones which contain an integrated vector; d) transiently transfecting the quantity of cell clones with a set of helper plasmids to produce a vector virus, wherein the set of helper plasmids contain a complement of structural genes which permit replication; e) infecting naïve cells from a cell culture with the vector virus; f) placing the cells from (e) under selection with the medium selectable for the selectable marker; g) cloning the cells from (f) to produce a quantity of cell clones, wherein each of the quantity of cell clones is designated as an Initiator Clone; h) confirming that the mutation target gene is functional in each Initiator Clone (IC) and, optionally, sequencing the mutation target gene for later comparison to mutation target genes which have undergone a cycle of replication; i) transiently transfecting the ICs with a set of helper plasmids to produce a vector virus; j) infecting naïve cells from a target cell culture with the vector virus from (i) to produce a quantity of infected target cells; k) placing a first portion of the quantity of infected target cells under selection with the medium selectable for the selectable marker and a substantially similar second portion of the quantity of infected target cells under selection with the medium selectable for the selectable marker plus a selective medium for the mutation target gene; and l) determining a viral titer for the first portion and a viral titer for the second portion by counting drug-resistant quantities, wherein the mutation frequency is calculated by dividing the viral titer of the second portion by the viral titer of the first portion, and the mutation rate is calculated by dividing the mutation frequency by the number of base pairs in the mutation target gene. In one embodiment, the assay further comprises providing a negative control group, wherein a quantity of naïve cells from the cell culture is left uninfected and placed under selection with the medium selectable for the selectable marker plus a medium selectable for the mutation target gene.

In more specific embodiments the retrovirus is selected from the group consisting of human immunodeficiency virus type 1, human immunodeficiency virus type 2, feline immunodeficiency virus, simian immunodeficiency virus, visna-maedi virus, caprine arthritis-encephalitis virus, equine infectious anemia virus, and bovine immunodeficiency virus. In a very specific embodiment the retrovirus is human immunodeficiency virus type 1.

In further specific embodiments the selectable marker gene comprises an antibiotic or drug resistance gene and the medium selectable for the selectable marker comprises the corresponding antibiotic or drug. In one aspect the selectable marker gene comprises a hygromycin B resistance gene and the medium selectable for the selectable marker comprises hygromycin B.

In additional embodiments the mutation target gene is a thymine kinase gene and the medium selectable for the mutation target gene comprises bromodeoxyuridine, gancyclivir, acyclivir, or HAT (hypoxanthine+aminopterin+thymine). In a very specific embodiment the medium selectable for the mutation target gene comprises bromodeoxyuridine.

In one aspect the cell culture comprises 143B cells. The 143B cell culture is a cell line available from the American Type Culture Collection, Manassas, Va. This cell line is a derivative of the human osteosarcoma cell line, HOS, and is negative for thymidine kinase (TK) function.

The following descriptions and examples of the various embodiments of the invention have been presented for the purposes of illustration and are not intended to be exhaustive or to limit the invention to the precise form disclosed. Many alternatives, modifications and variations will be apparent to those skilled in the art. Accordingly, this invention is intended to embrace all alternatives, modifications and variations that are discussed and exemplified herein, and others that fall within the spirit and broad scope of the claims.

### EXAMPLES

With respect to all the examples disclosed herein, the full-length HIV-1 genome that served as the basis for the HIV-1 vector (FIG. 1, top) and the p83-10 plasmid that contains the 3′ half of HIV-1_{NL4-3} were obtained through the AIDS Research and Reference Reagent Program, Division of AIDS, NIAID, NIH: pNL4-3. The helper plasmids, pCMVΔR8.2 and pMD.G, were generously provided from the University of Geneva, Switzerland. Plasmid pCMVAR8.2 encodes structural proteins Gag and Pol, in addition to all of the HIV-1 accessory proteins. This Gag-Pol helper expresses sequences derived from both the HXB2D and NILA-3 strains of HIV-1. However, the reverse transcriptase portion of pCMVΔR8.2 was derived completely from NL4-3 (accession no. M19921). The pMD.G helper plasmid expresses G glycoprotein of vesicular stomatitis virus (VSV.G) and confers broad tropism via pseudotyping of the HIV-1 vector virus. Both of the helper plasmids express genes from heterologous promoters. The pIREShyg2 plasmid was purchased from Clontech, a division of BD Biosciences, Palo Alto, Calif. The gene for thymidine kinase (tk) was cloned from plasmid pKOS 17B2, obtained from Harvard Medical School, Boston, Mass.

General molecular biology procedures, including restriction enzyme digestion, agarose gel electrophoresis, DNA ligation, and bacterial transformation were performed using standard published procedures (see Ausubel et al. “Current Protocols in Molecular Biology, John Wiley & Sons (1995) and Sambrook et al. “Molecular Cloning: a laboratory manual” Cold Spring Harbor Laboratory, NY, (1989), incorporated herein by reference).

All enzymes were purchased from New England Biolabs, Inc., Beverly, Mass., unless otherwise noted.

### Example 1

This example illustrates the difference in the mutation rate derived from employment of an embodiment of the inventive assay wherein HIV-1 is the retrovirus measured, and that derived from the reference Mansky in vivo assay on HIV-1.

The average mutation rate derived from the present assay is 1.6-fold lower than the rate reported by Mansky and Temin (1995) using the indirect in vivo assay for HIV-1 (Table 3). Mansky's average rate was 3.5×10⁻⁵ mutations/base/cycle. As shown in Table 3, Mansky's rate was based on lower numbers of mutants detected (specifically, 9.2-fold lower). Most likely, the lower frequency was due to the difference in the sizes of the mutational target sequences; the Mansky assay uses the lacZa peptide sequence, which is 3.6 times smaller than the tk sequence used in the practice of this embodiment of the invention. The number of viruses screened cannot be directly compared because this number is unknown for the Mansky assay. A total number of 15,930 viruses were screened in this example, while a total number of 8,678 bacterial colonies were screened in the Mansky assay. It is unlikely that each bacterial colony in the Mansky assay corresponds to an independent proviral sequence because cell colonies, each containing numerous cells, were pooled in the final step.

This last point may explain why differences were found in the types of mutations observed in the present example versus Mansky assays (Table 2). In both assays, the majority of mutations were base substitutions. However, there were significantly less G→A transitions observed in the present example as compared to the Mansky assay (p=0.04). There were significantly more insertions observed in the present example as compared to the Mansky assay (p=0.02). There were significantly less single nucleotide deletions observed in the present example as compared to the Mansky assay (p=0.02). Lastly, there were significantly less transition mutations, and conversely more transversion mutations, observed in the present assay as compared to the Mansky assay (p=0.01). Frequencies were used in a one-tailed Fisher's exact test to determine the statistical significance with an alpha level of 0.05. For some categories of mutants, the numbers were too small for statistical analyses.

### Example 2

This example illustrates construction of an HIV-1 vector, named pNL4-3Δ+cass.

A replication-defective HIV-1 vector was constructed based on the NL4-3 strain of HIV-1 (FIG. 1, top). The vector contains all of the cis-acting sequences necessary for replication, plus a complete set of intact accessory genes for HIV-1. Deletions were made in the gag, pol, and env genes to render the vector replication defective. Structural gene products, Gag, Pol, and Env, were supplied in trans by helper plasmids. In place of env, a mutational cassette was inserted that contained the human CMV promoter driving expression of tk, which provided the mutational target sequence for the assay. A functional tk gene renders cells resistant to medium containing HAT and susceptible to medium containing BrdU. An IRES sequence following tk allowed for expression of hyg, which provided a selectable marker via resistance to the antibiotic hygromycin B.

The final HIV-1 vector, named pNL4-3Δ+cass, was verified by restriction enzyme analysis, sequencing, and phenotypic testing in 143B cells. All plasmid constructs leading up to the final vector were digested with restriction enzymes to verify the cloning process. The final vector was digested with six restriction enzymes (Afl II, Hind III, Kpn I, Pst I, Sac I, and Xmn I) and showed only bands of the predicted sizes, indicating that no major rearrangements, insertions, or deletions had occurred during the cloning process (data not shown). Sequencing revealed that the tk gene had no mutational defects. The final vector was transfected into naïve 143B cells, which are negative for TK function. The transfected cells were resistant to medium containing HAT and susceptible to medium containing BrdU, indicating that tk was functional. The transfected cells were also resistant to medium containing hygromycin B, indicating that hyg was functional. Finally, transfection of naïve 143B cells with pNL4-3Δ+cass plus helper plasmids yielded virus, as indicated by successful transduction of the tk and hyg genes into fresh 143B cells.

To create the pNL4-3Δ+cass vector (FIG. 1, top), deletions were made in the gag, pol, and env genes of pNL4-3, and a mutational cassette was inserted at the location of the env deletion. The deletion in gag-pol (corresponding to nucleotides 1340-3716 of the original NL4-3 sequence, accession no. M19921) was created by digesting pNL4-3 with Swa I and purifying the 12,503 bp fragment, which contained the plasmid backbone and the gag-pol-deleted HIV-1 genome. The fragment was isolated using the Qiaquick Gel Extraction Kit (Qiagen, Valencia, Calif.) and ligated with T4 DNA ligase. This intermediate construct was named pNL4-3Δswa.

The deletion in env (corresponding to nucleotides 6401-7252 of the original NL4-3 sequence, accession no. M19921) was created in p83-10 (6,253 bp), which contains the 3′ half of the HIV-1 genome. Working with the 3′ half of the HIV-1 genome allowed the use of restriction enzymes that were not unique in the full-length pNL4-3 plasmid. Plasmid p83-10 was digested with Nde I and Nhe I; the 5,402 bp fragment, which contained the plasmid backbone and the env-deleted 3′ half of HIV-1, was isolated by using the Qiaquick Gel Extraction Kit (Qiagen). This fragment was ligated with complementary oligonucleotides that inserted a multiple cloning site (MCS), creating intermediate construct p83-10MCS. The sequences of the oligonucleotides are as follows: MCS1 5′-TAT GGG CGC GCC ACG CGT CCC GGG G-3′ and MCS2 5′-GCT AGC CCC GGG ACG CGT GGC GCG CCC ATA TG-3′. Restriction enzymes Sal I and Xho I were used to isolate a fragment from p83-10MCS that contained the env deletion. This fragment was then used to replace the corresponding fragment in pNL4-3Δswa that contained the intact env sequence. These cloning steps created intermediate construct pNL4-3Δ.

The mutational cassette was created by using pIREShyg2 from Clontech. The pIREShyg2 plasmid is a bi-cistronic expression vector that expresses a gene of interest (tk, in this case) from the human cytomegalovirus (CMV) promoter. An internal ribosome entry site (IRES) allows for expression of the second gene, hygromycin phosphotransferase (hyg), which provided a selectable marker via resistance to the antibiotic hygromycin B. The tk sequence was amplified by polymerase chain reaction (PCR) from plasmid pKOS17B2 using a set of primers containing BsrG I and BsiWI sites at 5′ and 3′ ends, respectively; tk was inserted into the MCS of pIREShyg2 using these sites after intermediary cloning in a TOPO TA vector (Invitrogen, Carlsbad, Calif.), creating intermediate construct pTKIREShyg. The sequence encompassing the CMV promoter, tk, IRES, and hyg was then amplified from pTKIREShyg using a set of primers containing Mlu I and Nhe I sites at 5′ and 3′ ends, respectively. Following intermediary cloning in a TOPO TA vector, these restriction sites were used to insert the CMV-tk-IRES-hyg mutational cassette into pNL4-3Δ to create the final vector, pNL4-3Δ+cass.

For cloning into the mutational cassette, the tk gene was amplified using Taq DNA polymerase in a 100-μl reaction, according to the manufacturer's protocol (Invitrogen). Twenty-five nanograms of plasmid pKOS17B2 was used in the reaction, along with 20 pmol of each of the following primers: TK1 5′-GCT ATG TAC AGC CAC CAT GCC CAC GCT ACT GCG GGT-3′ and TK2 5′-GTA CCG TAC GTC AGT TAG CCT CCC CCA TCT-3′. The TK1 primer encodes a BsrG I site near the 5′ end followed by a Kozak sequence. The TK2 primer encodes the 3′ end of tk, followed by a BsiWI site. Twenty-five cycles of the following program were performed: 15 sec. at 94° C., 15 sec. at 60° C., and 1 min. at 72° C. Taq DNA polymerase was added to the reaction after an initial heating step at 94° C. for 5 min, and a final extension step was performed at 72° C. for 10 min.

For cloning into the HIV-1 vector, the CMV-tk-IRES-tyg mutational cassette was amplified using PfuTurbo® polymerase (Stratagene, La Jolla, Calif.) in a 50-μl reaction, according to the manufacturer's protocol. Twenty-five nanograms of plasmid pTKIREShyg were used in the reaction, along with 10 pmol of each of the following primers: CMVCAS 1 5′-GAT CAC GCG TCG CGT TAC ATA ACT TAC GGT A-3′ and CMVCAS 2 5′-GTC AGC TAG CTT CCT TTG CCC TCG GAC GAG-3′. CMVCAS 1 encodes an Mlu I restriction enzyme site prior to the CMV promoter sequence, and CMVCAS 2 encodes the 3′ end of hyg, followed by a Nhe I restriction enzyme site. Twenty-five cycles of the following program were performed: 1 min. at 95° C., 1 min. at 58° C., and 4 min. at 72° C. An initial heating step at 95° C. for 3 min. and a final extension step at 72° C. for 10 min. were performed.

### Example 3

The following example illustrates utilization of one embodiment of the assay to determine the mutation rate of HV-1.

The mutation rate assay for this example is outlined in FIG. 1. Serial dilutions of HIV-1 vector DNA were transfected into 143B cells, which were placed in medium containing hygromycin B to select for resistant cells. Hygromycin B resistant cells were cloned by picking well-isolated colonies from plates receiving the highest dilutions of DNA (step 1, FIG. 1). In order to remove mutations that may have occurred during transfection and to improve the probability for isolating clones that contained only one vector per cell, a subsequent infection step was performed. The 143B clones containing the HIV-1 vector were transiently transfected with HIV-1 helper plasmids for transient production of vector virus (step 2, FIG. 1). Serial dilutions of this virus were then used to infect fresh 143B cells, which were placed in medium containing hygromycin B to select for resistant cells. Hygromycin B-resistant cells were cloned by picking well-isolated colonies from plates receiving the highest dilutions of virus (step 3, FIG. 1). The resulting cell clones were called the Initiator Cells (ICs) and served as the basis for the single cycle of replication assay.

The ICs generated in step 3, FIG. 1, were rigorously characterized by molecular and phenotypic analyses to ensure a successful and accurate mutation rate assay. Polymerase chain reaction was performed with the genomic DNA of each IC to detect the presence of provirus using primers specific for the sequence that spans CMV through tk. The same PCR was performed with the genomic DNA of naïve 143B cells as a negative control. The predicted PCR product of 1,600 bp in length was detected in all ICs and not in the naïve 143B cells (FIG. 2A). A Southern blot analysis was performed on the genomic DNA of each IC to ensure that each IC contained only one HIV-1 vector sequence. The cellular DNA was digested with EcoR I, which generated a 3′ fragment of the viral genome with adjacent cellular DNA. A radiolabeled probe specific for this 3′ region of the virus was used to detect a single band in each IC (FIG. 2B). The bands for each IC were different sizes, confirming that each IC was an independent cell clone (FIG. 2B). Each IC was grown in medium containing BrdU for a two-week period. The BrdU selection resulted in the complete death of ICs 2, 3, and 4, indicating functional tk expression in these cell clones. These ICs were chosen for the assay and their tk genes were sequenced and found to be identical to the tk sequence from the original pNL4-3Δ+cass vector. Transient transfection of helper plasmids into the ICs produced viral titers in the range of 10² to 10⁴cfu/ml, which provided a sufficient number of mutants from a practical number of infections.

Once the ICs were established, they were transiently transfected with the HIV-1 helper plasmids to generate virus for the single cycle of replication (step 4, 628 FIG. 1). HIV-1 vector virus was harvested from the ICs and used to infect fresh 143B cells (step 5, FIG. 1). Steps 4 and 5 constitute the single cycle of replication (from provirus to provirus, IC to target cell). Finally, parallel sets of infections were placed under two types of drug selection: hygromycin B plus BrdU (Hyg+BrdU) and hygromycin B alone (Hyg) (step 6, FIG. 1). Mock infections, performed in parallel as a negative control, showed no colony formation under the Hyg+BrdU selection. Viral titers were determined by counting the number of drug-resistant colonies in the linear range of the titration. The Hyg+BrdU titer revealed the number of mutants, while the Hyg titer revealed the number of viruses screened. The mutation frequency was calculated by dividing the total number of mutants detected by the total number of viruses screened (final step, FIG. 1 and Table 1).

For verification of the Initiator Clones, the CMV-tk region (1,600 bp) was amplified from genomic DNA (from step 3, FIG. 1) using Taq DNA polymerase (Invitrogen) in a 100-μl reaction, according to the manufacturer's protocol. Two hundred nanograms of genomic DNA were used in the reaction, along with 20 pmol of primers CMVCAS 1 and TK2. Twenty-five cycles of the following program were performed: 30 sec. at 94° C., 30 sec. at 58° C., and 2 min. at 72° C. Taq DNA polymerase was added to the reaction after an initial heating step at 94° C. for 5 min, and a final extension step was performed at 72° C. for 10 min.

The DNeasy Tissue Kit (Qiagen) was used to isolate the genomic DNA from cultured cells per manufacturer's protocol.

The 143B cell line was purchased from the American Type Culture Collection, Manassas, Va. This cell line is a derivative of the human osteosarcoma cell line, HOS, and is negative for thymidine kinase (TK) function. The cells were maintained in minimal essential medium (MEM) with Earl's BSS (Mediumtech, Herndon, Va.) supplemented with 10% fetal bovine serum, 2 mM L-glutamine, 100 U/ml penicillin, 100 μg/ml streptomycin (Invitrogen). Cells were passaged twice per week and were grown at 37° C. and 5% CO₂.

For selection of drug resistant colonies, cells were cultured for two weeks in medium containing the appropriate agent. The final concentration for each agent was determined empirically using 143B cells. The antibiotic hygromycin B (Invitrogen) was used at 0.27 mg/ml. The nucleoside analog bromodeoxyuridine (BrdU; Sigma-Aldrich Co., St. Louis, Mo.) was used at 0.12 mg/ml. The final concentrations of hypoxanthine, aminopterin, and thymine (HAT; Sigma-Aldrich) were 5×10⁻⁵ M, 2×10⁻⁷ M, and 8×10⁻⁶ M, respectively.

Cells were cloned by choosing well-isolated colonies from plates receiving the highest dilutions of DNA or virus (steps 1 and 3, respectively, FIG. 1) or plates in the linear range of the titration (step 6, FIG. 1). A sterile 5-mm Bel-Art cloning disk (Fisher Scientific, Pittsburgh, Pa.) treated with trypsin-EDTA (0.25%, Invitrogen) was placed onto the colony, incubated for 3½ min., and transferred into the well of a 24-well plate containing medium.

Stable and transient transfections (steps 1 and 2, FIG. 1) were performed by the dimethyl sulfoxide (DMSO)/polybrene method. For step 1, serial 5-fold dilutions of DNA, starting with 5 μg, were transfected onto 143B cells plated at 2×10⁵ cells/60 mm dish 24 hours pre-infection. For step 2, 9 μg of pCMVΔR8.2 and 3 μg of pMD.G were transfected onto cell clones (isolated from step 1) plated at 2×10⁵ cells/60-mm dish, 24 hours pre-transfetion. After 6 hours of incubation with DNA, cells were shocked with 25% DMSO for 3½ min. and were placed in appropriate selective medium at 24 hours post-transfection.

Transient transfection (step 4, FIG. 1) was performed using the LipofectAMINE™ 2000 per manufacturer's protocol (Invitrogen). Three micrograms of pCMVΔR8.2 and one microgram of pMD.G were transfected onto ICs (isolated from step 3) plated at 2×10⁵ cells/60-mm dish, 24 hours pre-transfection. The medium was replaced 24 hours after transfection.

Virus stock was harvested at 62 hours post-transfection by centrifugation of cell supernatant fluid at 1430×g for 10 min. at room temperature in sealed GH 3.8 rotor buckets of a Beckman GS-6R tabletop centrifuge to remove any detached cells. The 62-hour time point was chosen empirically based on transduction efficiencies with the HIV-1 based vector pHR'CMV-GFP, which encodes the gene for green fluorescent protein, and helper plasmids pCMVΔR8.2 and pMD.G.

Infections were performed with fresh virus stock and polybrene (8 μg/ml) in a final volume of 2 ml with cells plated 24 hours pre-infection at a density of 2×10⁵ cells/60-mm dish. Cells were incubated with virus for 3 hours, at which time the viral supernatant was replaced with fresh medium. At 24 hours post-infection, the medium was replaced with fresh medium containing the appropriate selective agent. Media containing the appropriate selective agents were replaced twice a week for two weeks. Serial 10-fold dilutions of virus were used to determine viral titers, which were measured by counting the number of drug-resistant colonies in the linear range of the titration. Viral titers are reported as the number of colony forming units per milliliter (cfu/ml).

Mutation frequencies and rates were calculated using simple mathematical formulas. The mutation frequency was calculated by dividing the total number of mutants by the total number of viruses (Hyg+BrdU titer/Hyg titer; FIG. 1 and Table 1). The mutation rate was calculated by dividing the mutation frequency by the number of bases in tk (996 bases).

### Example 4

This example illustrates determination of mutation types in the mutational target gene subsequent to the assay.

For sequencing of the final mutants, the tk gene was amplified from genomic DNA isolated from the Target Cells (final step, FIG. 1) using Accuprime™ Taq DNA polymerase (Invitrogen) in a 10-μl reaction, according to the manufacturer's protocol. Three hundred nanograms of genomic DNA were used in the reaction, along with 20 pmol of each of the following primers: TKA1 5′-TCA CTA TAG GGA GAC CCA AGC 3′ and TKA2 5′-CCC TCG CAG ACA GCG AAT TAA-3′. Twenty-five cycles of the following program were performed: 45 sec. at 94° C., 45 sec. at 58° C., and 1 min. at 72° C. An initial heating step at 94° C. for 2 min. and a final extension step at 72° C. for 10 min. were performed. Primers and excess nucleotides were inactivated by adding 1.9 U Exonuclease I and 0.38 U Shrimp Alkaline Phosphatase (Amersham Biosciences, Piscataway, N.J.) to the PCR reaction. The final reaction volume was adjusted to 12.5 μl using ultra pure water. The reaction was incubated at 37° C. for 30 min., and enzymes were heat inactivated at 80° C. for 20 min.

All PCR reactions were performed on a Peltier Thermal Cycler 200 (MJ Research, Reno, Nev.) with the exception of the final amplification of tk from the Target Cells, which was performed on a GeneAmp PCR System 9600 (Applied Biosystems, Foster City, Calif.).

Southern blotting was performed using standard published procedures (Ausubel and Sambrook, supra). Briefly, 15 μg of genomic DNA was digested with EcoR I and separated in a 1% agarose gel. DNA was transferred via capillary action onto a Hybond-N⁺ membrane (Amersham). The membrane was hybridized with a radiolabeled fragment complementary to the 3′ half of the HIV-1 genome (a 422-bp BamH I to Xho I fragment, specific for the Rev-responsive element and tat gene). The fragment was labeled using the Rediprime™ II DNA Labeling System and α-³²P dCTP per manufacturer's protocol (Amersham). The Qiaquick Nucleotide Removal Kit (Qiagen) was used to remove the excess dNTPs from the radiolabeled probe. The radioactive content of the probe was quantified using a scintillation analyzer. Results were visualized by autoradiography using Kodak X-OMAT AR2 film (Fisher Scientific).

Purified PCR products were reduced to a concentration of 50 ng/μl by centrifugation under vacuum, and 200 ng of each sample was sent to Lark Technologies, Inc., Houston, Tex. for sequencing. The following two primers were used: FORWARD-TK 5′-TAC CTT ATG GGC AGC ATG ACC-3′ and REVERSE-TK 5′-CTG CAG ATA CCG CAC CGT ATT-3′. The sequencing results were aligned and analyzed for non-synonymous base substitutions, insertions, and deletions using MacVector™ software version 6.5.3 (Accelrys, San Diego, Calif.).

### Example 5

This example compares the mutation rates calculated according to an embodiment of the present assay with calculations reported for a widely known assay that also employs tk as the mutational target.

The overall mutation rate derived according to the present inventive assay for HIV-1 was slightly higher and the tk inactivation rate was slightly lower in comparison to an in vivo study on Moloney murine leukemia virus (MoMLV), in which tk was part of a larger mutational cassette. In the MoMLV study, an overall mutation rate of 1.6×10⁻⁵/base/cycle and a tk inactivation rate of 3.0% per kbp were reported. One difference is the types of mutations observed. The majority of mutations detected in the mutational target gene of the present assay were base substitutions, while the majority of mutations in the MoMLV study were labeled as “gross rearrangements” (deletions, deletions with insertions, duplications, and complex hypermutations). The majority of gross rearrangements in the MoMLV study were found at locations corresponding to open regions of RNA: hairpin loops, internal loops, and bulges. This underscores the importance of sequence context in interpreting patterns of mutations. The different patterns of mutation observed between MoMLV (a γ-retrovirus) and HIV-1 (a lentivirus) also emphasizes the caution that must be exercised when extrapolating results from one retroviral genus to another.

### Examplre 6

The following example illustrates calculation of the mutation rate.

Mutation rates were calculated from 27 independent infections from each IC (FIG. 3 and Table 1). Mock infections, performed in parallel as a negative control, showed no colony formation under Hyg+BrdU selection. The mutation rate was calculated by dividing the mutation frequency (Table 1) by the size of the tk sequence, which was 996 bases from start to stop codon. The average mutation rate for HIV-1 was 2.2×10⁻⁵ mutations/base/cycle of replication (Table 1).

### Example 7

This example illustrates determination of the types of mutations that resulted in loss of TK function.

Twenty-seven of 349 mutants were randomly selected for sequencing in order to determine the types of mutations that resulted in loss of function for TK (Table 2). The majority of mutations (65%) were base substitutions with a preponderance of C→U (21%) and G→A (14%) mutations. One G→A and one C→A hypermutant were observed, as defined by nonconsecutive but repetitive substitution of adenosines. In this case, the hypermutation was not extensive; only two or three substitutions were observed in each hypennutant. Insertions were observed approximately twice as often as deletions (23% and 12%, respectively). Among the insertions, a predominance of single adenosine additions was observed at a given time in an infected individual. Thus, such a hypermutaion could predict the future pattern of mutation and could assist in making treatment decisions, especially for newly infected people and those who are changing drugs due to treatment failure.

TABLE 1

| | IC2 | IC3 | IC4 | Totals |
|---|---|---|---|---|
| Number of mutants detected | 142 | 53 | 154 | 349 |
| Number of viruses screened | 5,265 | 3,375 | 7,290 | 15,930 |
| Mutation frequency^{a} | 2.7 | 1.6 | 2.1 | 2.2 |
| (×10⁻² mutations/cycle) | | | | |
| Mutation rate^{b} | 2.7 | 1.6 | 2.1 | 2.2 |
| (×10⁻⁵ mutations/base/cycle) | | | | |

Mutation data for individual Initiator Clones (IC)
^{a}Mutation frequency = Number of mutants detected/Number of viruses screened
^{b}Mutation rate = Mutation frequency/Number of bases in tk gene (996 bases)

TABLE 2
Types of mutations observed

No. of mutations observed (%)

| Type of mutation^{a} | Present Assay | Mansky Assay^{b} |
|---|---|---|
| Substitutions | | |
| C → U | 9 (21) | 6 (15) |
| * G → A | 6 (14) | 13 (33) |
| U → A | 4 (9) | 1 (3) |
| C → A | 2 (5) | 0 |
| A → G | 1 (2) | 0 |
| G → U | 1 (2) | 0 |
| U → G | 0 | 1 (3) |
| U → C | 0 | 2 (5) |
| G → A hypermutant^{c} | 1 [+2 bases] (2) | 2 [+2 bases] (3) |
| C → A hypermutant^{c} | 1 [+ 3 bases] (2) | 0 |
| Total | 28 (65) | 27 (68) |
| Insertions | | |
| +A | 6 (14) | 1 (3) |
| +G | 1 (2) | 0 |
| +U | 3 (7) | 1 (3) |
| * Total | 10 (23) | 2 (5) |
| Deletions | | |
| Δ33 | 0 | 1 (3) |
| Δ21 | 0 | 1 (3) |
| Δ11 | 1 (2) | 0 |
| Δ10 | 1 (2) | 0 |
| Δ8 | 0 | 1 (3) |
| Δ5 | 1 (2) | 0 |
| Δ2 | 1 (2) | 0 |
| * Δ1 | 1 (2) | 7 (18) |
| Total | 5 (12) | 10 (25) |
| Deletion with Insertion | | |
| Δ4, +15 | 0 | 1 (3) |
| Total Mutations | 43 (100) | 40 (100) |
| Total Mutants Screened | 27 [26,892 bases] | 38 [10,640 bases] |

^{a}The asterisk indicates statistically significant differences between the present and Mansky assays, as determined using a one-tailed Fisher's Exact Test.
^{b}Mansky and Temn, 1995.
^{c}Each mutation counted separately toward the total number of mutations.

TABLE 3
Comparison of mutation data between assays

| | Present Assay | Mansky Assay^{a} |
|---|---|---|
| Number of mutants | 349 | 38^{b} |
| detected | | |
| Number of viruses screened | 15,930 | 8,678^{c} |
| Mutation frequency^{d} | 2.2 | 4.4 |
| (×10⁻² mutations/cycle) | | |
| Mutation rate^{e} | 2.2 | 3.5 |
| (×10⁻⁵ mutations/base/cycle) | | |

^{a}Mansky and Temin, 1995
^{b}Interpreted from the number of mutant bacterial colonies
^{c}Interpreted from the total number of bacterial colonies
^{d}Mutation frequency = Number of mutants detected/Number of viruses screened
^{e}Mutation rate = Mutation frequency/Number of bases in each target sequence

1. A lentivirus-based retroviral vector wherein the vector comprises: at least a portion of a lentivirus genome; a disrupted gag and a disrupted pol gene such that the vector is rendered replication-defective; a disrupted env gene; and a mutational cassette, wherein the mutational cassette comprises sequences encoding: a mutation target promoter sequence; a genomic source comprising a mutation target gene wherein there is a number of base pairs in the mutation target gene; an internal ribosome entry site for expression of a selectable marker gene; and a selectable marker gene.

2. The vector recited in claim 1 wherein the vector is transduction- and infection-competent.

3. The vector recited in claim 1 wherein the lentivirus genome is selected from the group consisting of human immunodeficiency virus type 1, human immunodeficiency virus type 2, feline immunodeficiency virus, simian immunodeficiency virus, visna-maedi virus, caprine arthritis-encephalitis virus, equine infectious anemia virus, bovine immune deficiency virus, and hybrids thereof.

4. The vector recited in claim 1 wherein the lentivirus genome comprises human immunodeficiency virus type 1.

5. The vector recited in claim 1 wherein the selectable marker comprises a positive selectable marker.

6. The vector recited in claim 1 wherein the selectable marker comprises a hygromycin B resistance gene.

7. The vector recited in claim 1 wherein the selectable marker comprises a hygromycin B phosphotransferase gene.

8. The vector recited in claim 1 wherein the mutation target gene comprises greater than 300 base pairs.

9. The vector recited in claim 1 wherein the mutation target gene comprises greater than 500 base pairs.

10. The vector recited in claim 1 wherein the mutation target gene comprises greater than 700 base pairs.

11. The vector recited in claim 1 wherein the mutation target promoter gene comprises a human cytomegalovirus promoter, and the mutation target comprises a thymidine kinase gene.

12. The vector recited in claim 1 wherein the genomic source comprising a mutation target gene comprises a human herpes virus type 1 gene.

13. A cell comprising the lentivirus-based retroviral vector as recited in claim 1.

14. The cell recited in claim 13 wherein the cell is a dividing or non-dividing eukaryotic cell.

15. The cell recited in claim 14 wherein the cell is a dividing cell.

16. The cell recited in claim 14 wherein the cell is a non-dividing cell.

17. An assay for determining a mutation frequency and a mutation rate of a retrovirus comprising:
a) constructing the vector recited in claim 1;
b) stably transfecting cells from a cell culture with the vector from (a)
c) placing the cells under selection with a medium selectable for the selectable marker to produce a quantity of cell clones which contain an integrated vector;
d) transiently transfecting the quantity of cell clones with a set of helper plasmids to produce a vector virus, wherein the set of helper plasmids contain a complement of structural genes which permit replication;
e) infecting naïve cells from a cell culture with the vector virus;
f) placing the cells from (e) under selection with the medium selectable for the selectable marker;
g) cloning the cells from (f) to produce a quantity of initiator cell clones, wherein each of the quantity of cell clones is designated as an Initiator Clone (IC);
h) confirming that the mutation target gene is functional in each IC and sequencing the mutation target gene for later comparison to mutation target genes which have undergone a cycle of replication;
i) transiently transfecting the Initiator Clones with a set of helper plasmids to produce a vector virus;
j) infecting naïve cells from a target cell culture with the vector virus from (i) to produce a quantity of infected target cells;
k) placing a first portion of the quantity of infected target cells under selection with the medium selectable for the selectable marker and a substantially similar second portion of the quantity of infected target cells under selection with the medium selectable for the selectable marker plus a selective medium for the mutation target gene; wherein a number of drug resistant colonies grows from the first and second portions, and
l) determining a viral titer for the first portion and a viral titer for the second portion by counting the number of drug-resistant colonies;
wherein the mutation frequency is calculated by dividing the viral titer of the second portion by the viral titer of the first portion, and the mutation rate is calculated by dividing the mutation frequency by the number of base pairs in the mutation target gene.

18. The assay as recited in claim 17 wherein the cell culture is negative for the selectable marker.

19. The assay as recited in claim 17 wherein the retrovirus is selected from the group consisting of human immunodeficiency virus type 1, human immunodeficiency virus type 2, feline immunodeficiency virus, simian immunodeficiency virus, visna-maedi virus, caprine arthritis-encephalitis virus, equine infectious anemia virus, and bovine immune deficiency virus, and hybrids thereof.

20. The assay as recited in claim 17 wherein the retrovirus is human immunodeficiency virus type 1.

21. The assay as recited in claim 17 wherein the selectable marker gene comprises a hygromycin B resistance gene and the medium selectable for the selectable marker comprises hygromycin B.

22. The assay as recited in claim 21 wherein the hygromycin B resistance gene comprises hygromycin B phosphotransferase gene.

23. The assay as recited in claim 17 wherein the mutation target gene is a thymidine kinase gene and the medium selectable for the mutation target gene comprises bromodeoxyuridine or HAT.

24. The assay as recited in claim 17 wherein the medium selectable for the mutation target gene comprises bromodeoxyuridine.

25. The assay as recited in claim 17 wherein the cell culture comprises 143B cells.

26. The assay as recited in claim 17 wherein the assay further comprises providing a negative control group, wherein a quantity of naïve cells from the target cell culture is left uninfected and placed under selection with a medium selectable for the selectable marker plus a medium selectable for the mutation target gene.
